(19) United States
(12) Plant Patent Application Publication
(10) Pub. No.: US 2010/0293690 P1
(43) Pub. Date: Nov. 18, 2010

(54) Lobelia plant named 'DANANAB8'

(50) ;

(76) Inventor: Gabriel Danziger, Beit Dagan (IL)

Correspondence Address: HortisUSA, PO Box 3109, Camarillo, CA, 93011, US

(21) Appl. No.: 12/454,554

(22) Filed: May 18, 2009

(51) Int. Cl. A01H 5/00

(52) U.S. Cl. PLT/451

(57) ABSTRACT

A new and distinct Lobelia cultivar named ‘DANANAB8’ is disclosed, characterized by having clear white flowers flowers, upright plant habit, early flowering, and a high temperature tolerance. The new variety is a Lobelia, normally produced as an outdoor garden or container plant.

### LATIN NAME OF THE GENUS AND SPECIES

Lobelia erinus

### VARIETY DENOMINATION

‘DANANAB8’

### BACKGROUND OF THE INVENTION

The new Lobelia cultivar is a product of a planned breeding program conducted by the inventor, Gabriel Danziger, in Moshav Mishmar Hashiva, Israel. The objective of the breeding program was to produce new varieties of Lobelia for commercial introduction. The seed parent is the unpatented, proprietary seedling variety referred to as Lobelia erinus ‘LOB-6-478.’ The pollen parent is the unpatented, proprietary pollen variety referred to as Lobelia erinus ‘LOB-6241.’ The new variety was discovered in April 2006 by the inventor in a group of seedlings resulting from that crossing, in a commercial greenhouse in Moshav Mishmar Hashiva, Israel. Asexual reproduction of the new cultivar ‘DANANAB8’ by vegetative cuttings was first performed at a commercial greenhouse in Moshav Mishmar Hashiva, Israel in July 2006. Subsequent propagation has shown that the unique features of this cultivar are stable and reproduced true to type on successive generations.

### SUMMARY OF THE INVENTION

The cultivar ‘DANANAB8’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DANANAB8.’ These characteristics in combination distinguish ‘DANANAB8’ as a new and distinct Lobelia cultivar:
1. Upright plant habit.
2. Contrasting flower coloration between the white and the blue.
3. Early blooming compared to similar Lobelia cultivars.

### PARENTAL COMPARISON

Plants of the new cultivar ‘DANANAB8’ are similar to plants of the seed parent, ‘LOB-6-478’ in most horticultural characteristics, however, plants of the new cultivar ‘DANANAB8’ have a darker color blue flower and more upright plant habit.

Plants of the new cultivar ‘DANANAB8’ are similar to plants of the pollen parent, ‘LOB-6241’ in most horticultural characteristics, however, plants of the new cultivar ‘DANANAB8’ have a different plant habit and different branching characteristics.

### COMMERCIAL COMPARISON

Plants of the new cultivar ‘DANANAB8’ can be compared to the commercial variety ‘DANANAB2,’ U.S. Plant Pat. No. 19,332. Plants of ‘DANANAB8’ are similar to plants of ‘DANANAB2, in most horticultural characteristics, however, plants of the new variety have an upright growth habit, compared to the trailing growth habit of ‘DANANB2.’

### BRIEF DESCRIPTION OF THE PHOTOGRAPH

The accompanying photograph in FIG. 1 illustrates in full color a typical plant of ‘DANANAB8’ grown in a greenhouse, in a 13 cm pot. Age of the plant photographed is approximately 6 weeks from a rooted cutting, with 2 rooted cuttings planted in the pot shown.

FIG. 2 illustrates in full color a close up of a typical bloom of ‘DANANAB8.’ The photographs were taken using conventional techniques and although colors may appear different from actual colors due to light reflectance it is as accurate as possible by conventional photographic techniques.

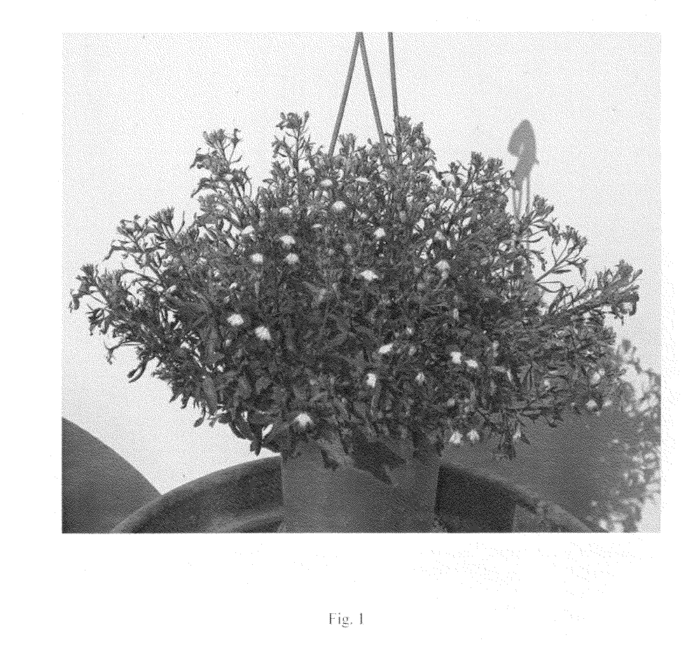

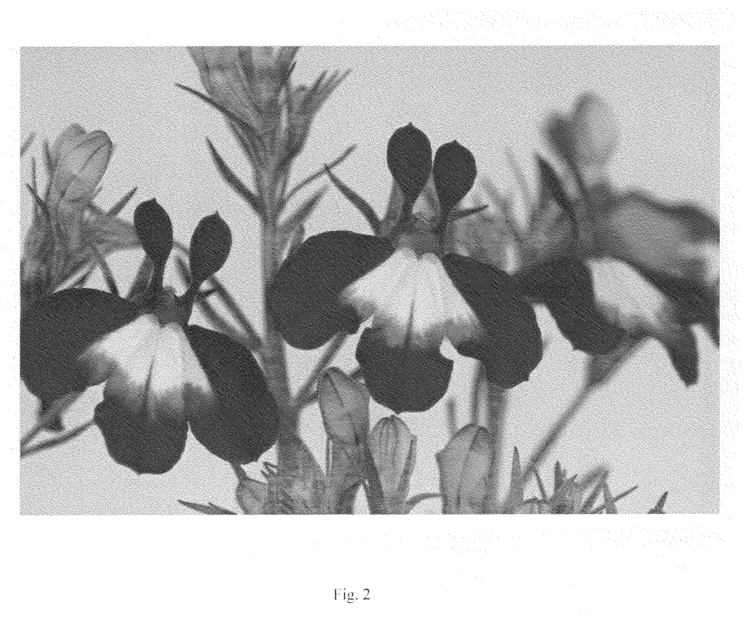

### DETAILED BOTANICAL DESCRIPTION

In the following description, color references are made to The Royal Horticultural Society Colour Chart except where general terms of ordinary dictionary significance are used. The following observations and measurements describe ‘DANANAB8’ plants grown in a greenhouse in Moshav Mishmar Hashiva, Israel. The growing temperature ranged from day temperatures of 18° C. to 25° C., and night temperatures of 12° C. to 18° C. A balanced fertilizer of 75 to 200 ppm Nitrogen was used. Day length of 11-14 hours. The greenhouse is un-shaded, giving bright, normal sunlight conditions. Measurements and numerical values represent averages of typical plant types.
Botanical classification: Lobelia erinus cultivar ‘DANANAB8.’
Propagation:
Time to initiate roots: About 8 to 14 days at approximately 25° C.
Root description: Fibrous.
Plant:
Growth habit: Mounding.
Pot size of plant described: 13 cm.
Height:
   To top of flowers.—Approximately 20 cm, measured from the soil level of the pot.
Plant spread: Approximately 30 cm.
Growth rate: Very fast.
Branching characteristics: Medium branching.
Length of lateral branches: Approximately 12-17 cm.
Diameter of lateral branches: Approximately 0.2 cm.
Color of branches: Near RHS Green 139 B.
Texture: Pubescent.
Internode length: Approximately 2.0 cm.
Age of plant described: Approximately 50 days from a rooted cutting.
Foliage:
Arrangement : Alternate.
Leaves per branch: Approximately 10.
Leaves:
   Size.—Length: About 1.8 cm Width: About 0.4 cm Shape of leaf: Serrate Shape of apex: Acute Shape of base: Cuneate Texture: Glabrous Aspect: 90° relative to the branch Margin type: Entire.
   Color.—Young leaves: Upper surface: Near RHS Green 139 A Lower surface: Near RHS Green 139 B Mature leaves: Upper surface: Near RHS Green 139 B Lower surface: Near RHS Green 139 C.
Petiole: No Petiole.
Veins:
   Venation pattern.—Pinnate.
   Color.—Upper surface: Near RHS Green 139 B Lower surface: Near RHS Green 139 B.
Flower:
Bud:
   Shape.—Flattened oval.
   Diameter.—Approximately 0.3-0.4 cm.
   Length.—Approximately 0.4 cm.
   Color.—Near RHS Yellow 1C.
Natural flowering season: Early Spring to fall in Israel.
Flower type and habit: Auxillary.
Corolla:
   Upper petals (upper lip).—Number: 5 Length: Central petals: 8-10 mm Lateral petals: 8 mm Width: Central petals: 6 mm Lateral petals: 5 mm Shape: Elliptic Aspect: 45 degrees to branch Margin: entire Texture: smooth Color: When opening: Upper surface: Near RHS Blue Group 105 A with white center Lower surface: Near RHS Blue Group 104 B with white center Fully opened: Upper surface: Near RHS Blue Group 104-B with white center Lower surface: Near RHS Blue Group 104-B with white center.
   Lower petal (lower lip).—Length: 1.5 cm Width: 0.8 cm Shape: close to reniform Apex shape: blunt Color: Upper surface: White N999D Lower surface: White N999D.
   Palate.—Length: 8 mm Width: 5 mm Color: Yellow 4B Texture: pubescent.
   Throat.—Length: 6 mm Width: 5 mm Texture: Smooth Pubescence: none.
   Spur.—Quantity: 1 Length: 4 mm Diameter: 1 mm Texture: glabrous Color: Yellow 4C.
Calyx:
   Form.—oblanceolate.
   Length.—Approximately 0.6 cm.
   Diameter.—Approximately 0.1 cm.
   Sepal.—Width: 1 mm Shape: Litgulate Apex: acute. Margin: Entire. Texture: Smooth. Color: Upper surface: Near RHS Green 139 B Lower surface: Near RHS Green 139 B.
Pedicel:
   Strength.—fragile.
   Aspect.—upright.
   Length.—8 mm.
   Diameter.—0.5 mm.
   Texture.—glabrous.
   Color.—Yellow-Green 144A.
Fragrance: None.
Reproductive organs:
Stamens:
   Number (per flower).—1.
   Filament length.—About 1 cm.
   Anthers.—Shape: Cylindrical Length: About 0.2 mm Color: Near RHS Black 202 B.
Pollen:
   Color.—Near RHS Yellow 11 B.
   Amount.—Scant.
Pistils:
   Quantity per flower.—1.
   Length.—About 1 cm.
   Styles.—Length: About 0.9 cm Color: Near RHS Yellow-Green N144 B.
   Stigma.—Shape: Disc Color: Near RHS Black 202A with white hair.
Other characteristics:
Disease resistance: Neither resistance nor susceptibility observed.
Temperature tolerance: The new variety tolerates temperatures between 5 to 35° C.
Seed and fruit production: Produces large amounts of seed.

What is claimed is:

1. A new and distinct cultivar of Lobelia plant named ‘DANANAB8’ as herein illustrated and described.
